(19) United States
(12) Patent Application Publication
(10) Pub. No.: US 2023/0043102 A1
(43) Pub. Date: Feb. 9, 2023

(54) PERITONEAL DIALYSIS CATHETER

(71) Applicant: Seamless Solutions LLC, Slingerlands, NY (US)

(72) Inventors: Vincent Carsillo, Slingerlands, NY (US); Michael Gallichio, Slingerlands, NY (US)

(21) Appl. No.: 17/756,448

(22) PCT Filed: Dec. 11, 2019
(86) PCT No.: PCT/US2019/065646

§ 371 Date: May 25, 2022

(51) Int. Cl. A61M 1/28; A61M 25/02

(52) U.S. Cl. CPC A61M 1/285; A61M 25/02; A61M 2025/0293

(57) ABSTRACT

A peritoneal dialysis (PD) catheter is disclosed. In various embodiments, the PD catheter includes an improved intraperitoneal drain array as well as an improved anchor for use in placement of the device.

### BACKGROUND OF THE INVENTION

The invention relates generally to peritoneal dialysis catheters and, more particularly, to a peritoneal dialysis catheter configured for use within 24-48 hours after insertion.

Patients suffering from end stage renal disease typically rely on hemodialysis or peritoneal dialysis procedures to perform the blood filtration work previously performed by healthy kidneys. In particular, this includes the filtration of wastes and extra fluid from the body, and balancing of salts and minerals in the blood, including calcium, phosphorus, sodium, and potassium.

In hemodialysis, blood is removed from the body for dialysis, pumped through a filter outside the body to extract, e.g., wastes and extra fluid, and returned to the body. In typical treatment regimens, hemodialysis may be performed by trained healthcare providers at a dialysis center on a set schedule that may be, e.g., about three times/week. Hemodialysis is a common first line treatment for patients diagnosed with, e.g., end stage renal disease, because it typically can be performed on an urgent basis, very soon after a medical need for dialysis is diagnosed.

Peritoneal dialysis offers an alternative to hemodialysis while accomplishing the same goal of removing waste and extra fluid from the bloodstream. In peritoneal dialysis, a dialysis solution or dialysate is infused into a patient's peritoneal cavity via a catheter that is semi-permanently implanted through the skin, subcutaneous fat, rectus muscle, and into the peritoneal cavity. The intraperitoneal portion of the catheter includes sidewall openings, often referred to as holes, through holes, perforations, or fenestrations, that are in fluid communication with the catheter central bore, and which allow the dialysate to flow between the catheter and the peritoneal cavity. While in the peritoneal cavity and over a period of hours, the dialysate contacts the patient's peritoneal membrane, and waste and excess water from the bloodstream are transferred across the osmotic gradient of the peritoneal membrane and into the dialysate. The spent dialysate, including breakdown products from the blood, is then drained from the peritoneal cavity via the catheter, taking the waste and excess water with it. This process is then repeated, either manually (as in continuous ambulatory peritoneal dialysis, or CAPD) or automatically (as in automated peritoneal dialysis, or APD) via a dialysis machine.

Although peritoneal dialysis provides quality of life advantages over hemodialysis, including a lower scheduling and logistical burden on the patient and the ability to perform dialysis procedures at a more frequent interval, many patients and providers elect to treat renal failure with hemodialysis. Prior peritoneal dialysis catheters such as, e.g., the Toronto Western, Tenckhoff, and Missouri catheters, may require a clinically significant healing time, for example of about two weeks, between the time of insertion and the first time the catheter may be used to perform dialysis to avoid undesired consequences including, e.g., leaking, infection, etc. Prior devices may include features such as, e.g., a substantially spherical or bead-shaped anchor, whose geometry complicates surgical placement in the rectus muscle. Since many patients present with acute illness and require dialysis much sooner than two weeks, many patients commence treatment with hemodialysis. Despite the significant lifestyle restrictions of hemodialysis, many patients opt not to make a second significant lifestyle shift to move to peritoneal dialysis. Additionally, the low in situ flow rate through previous catheters results in longer exchange times than ideal.

### BRIEF DESCRIPTION OF THE INVENTION

A first aspect of the disclosure provides a catheter comprising: an intraperitoneal tubing segment including a drain array, the drain array including a plurality of holes through a wall of a distal end of the intraperitoneal tubing segment, a subcutaneous tubing segment in fluid connection with a proximal end of the intraperitoneal tubing segment; and an extraperitoneal tubing segment in fluid connection at a distal end thereof with a proximal end of the subcutaneous tubing segment, wherein a proximal end of the extraperitoneal tubing segment is configured for connection to a transfer set. A first cuff is disposed about the catheter between the subcutaneous tubing segment and the extraperitoneal tubing segment; and a second cuff is disposed about the catheter between the subcutaneous tubing segment and the intraperitoneal tubing segment. A flange is provided disposed about the catheter and adjacent to a distal end of the second cuff; as well as an anchor including: a frustoconical neck member having a base, a radially outward facing surface, and a planar surface opposite the base, the frustoconical neck member being positioned such that the planar surface opposite the base is adjacent and substantially parallel to the distal side of the flange, and a bulbous member coupled to the base of the frustum of the frustoconical neck member.

A second aspect of the disclosure provides an anchor for use in a semi-permanent implantable catheter, comprising: a frustoconical neck member having a base, a radially outward facing surface, and a planar surface opposite the base, and a bulbous member coupled to the base of the frustoconical neck member.

A third aspect of the disclosure provides a catheter comprising: an intraperitoneal tubing segment including a drain array, the drain array including a plurality of holes through a wall of a distal end of the intraperitoneal tubing segment, a subcutaneous tubing segment in fluid connection with a proximal end of the intraperitoneal tubing segment; and an extraperitoneal tubing segment in fluid connection at a distal end thereof with a proximal end of the subcutaneous tubing segment, wherein a proximal end of the extraperitoneal tubing segment is configured for connection to a transfer set. A first cuff is disposed about the catheter between the subcutaneous tubing segment and the extraperitoneal tubing segment; and a second cuff is disposed about the catheter between the subcutaneous tubing segment and the intraperitoneal tubing segment. A flange is provided and disposed about the catheter and adjacent to a distal end of the second cuff; as well as an anchor adjacent to the distal side of the flange. The catheter may be 5 mm diameter tubing with a 0.9 mm wall rendering a 3.2 mm lumen, and the drain array in the intraperitoneal tubing segment may include about sixty through holes.

These and other aspects, advantages and salient features of the invention will become apparent from the following detailed description, which, when taken in conjunction with the annexed drawings, where like parts are designated by like reference characters throughout the drawings, disclose embodiments of the invention.

### BRIEF DESCRIPTION OF THE DRAWINGS

FIG. 1 provides a side view of a peritoneal dialysis catheter, according to an embodiment of the invention.

FIG. 2 provides an enlarged side view of an anchor as deployed in the peritoneal dialysis catheter of FIG. 1, according to an embodiment of the invention.

FIG. 3 provides a proximal end view of the peritoneal dialysis catheter of FIG. 1 including a cuff, according to an embodiment of the invention.

FIG. 4 provides a distal end view of the peritoneal dialysis catheter of FIG. 1 including an anchor and flange, according to an embodiment of the invention.

FIG. 5 provides a perspective view of a peritoneal dialysis catheter, according to an embodiment of the invention.

FIG. 6 provides an end front view of an intraperitoneal tubing segment of a peritoneal dialysis catheter, according to an embodiment of the invention.

FIG. 7 provides a perspective view of a portion of an intraperitoneal tubing segment of a peritoneal dialysis catheter, according to an embodiment of the invention.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE INVENTION

Embodiments of the invention are described below, providing an improved catheter for use in peritoneal dialysis. At least one embodiment of the present invention is described below in reference to a nominal size and including a set of nominal dimensions. It should be apparent to those skilled in the art that the present invention is likewise applicable to various scales of the nominal size and/or nominal dimensions, for example, to accommodate patients of varying physical sizes, and variations in placement of the device.

As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quantity). The suffix “(s)” as used herein is intended to include both the singular and the plural of the term that it modifies, thereby including one or more of that term (e.g., the metal(s) includes one or more metals). Ranges disclosed herein are inclusive and independently combinable (e.g., ranges of “up to about 25 mm, or, more specifically, about 5 mm to about 20 mm,” is inclusive of the endpoints and all intermediate values of the ranges of “about 5 mm to about 25 mm,” etc.).

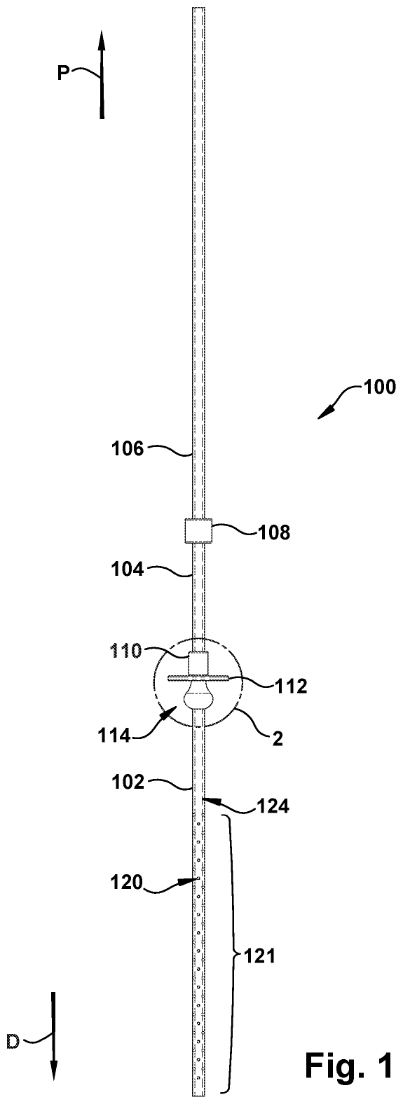

With reference to FIG. 1, in an embodiment, a catheter 100 is provided for use in performing peritoneal dialysis. Catheter 100 includes an intraperitoneal tubing segment 102, a subcutaneous tubing segment 104, and an extraperitoneal tubing segment 106. References to intraperitoneal tubing segment 102, subcutaneous tubing segment 104, and extraperitoneal tubing segment 106 may identify segments or sections of a single continuous length of tubing that makes up catheter 100, or may alternatively be separate lengths of tubing that are coupled together such that the central bores of each segment are substantially aligned and are in fluid connection with one another.

Subcutaneous tubing segment 104 is disposed between intraperitoneal tubing segment 102 on a distal end (D), and extraperitoneal tubing segment 106 on a proximal end (P) thereof, such that the proximal end of intraperitoneal tubing segment 102 is coupled to a distal end of subcutaneous tubing segment 104, and the proximal end of subcutaneous tubing segment 104 is coupled to the distal end of extraperitoneal tubing segment 106. Intraperitoneal tubing segment 102 and subcutaneous tubing segment 104 are configured for insertion into a patient for use in performing peritoneal dialysis procedures. When intraperitoneal tubing segment 102 and subcutaneous tubing segment 104 are inserted, extraperitoneal tubing segment 106 extends outside the patient's body, and is configured for connection to a transfer set as understood by one of skill in the art for use in performing peritoneal dialysis procedures. In various embodiments, intraperitoneal tubing segment 102 may have a length of about 160 mm±10 to 15 mm, and subcutaneous tubing segment 104 may have a length of about 45 mm±10 to 15 mm, although other lengths are also contemplated, and may be desired, particularly for use in patient populations with divergent needs.

Catheter 100 further includes two substantially annularly shaped cuffs 108, 110. A first cuff 108 is disposed about the catheter 100, defining the transition between the subcutaneous tubing segment 104 and the extraperitoneal tubing segment 106. A second cuff 110 is disposed about the catheter 100, defining the transition between the subcutaneous member 104 and the intraperitoneal member 102. Each of first cuff 108 and second cuff 110 are adhered to the outer circumference of catheter 100 to affix and maintain their axial positions with respect to catheter 100. Adhesives such as, e.g., methyl 2-cyanoacrylate (MCA), ethyl 2-cyanoacrylate, octyl cyanoacrylate, 2-octyl cyanoacrylate or any other suitable material having similar adhesive characteristics may be used.

First cuff 108 and second cuff 110 may be referred to as “superficial” and “deep” cuffs, respectively, referring to the positions of the respective cuffs when the catheter is inserted into a patient. In particular, when inserted, first cuff 108 is relatively superficially embedded beneath the epidermis, while second cuff 110 is embedded more deeply, within the patient's rectus muscle. When inserted and during use, the subcutaneous tubing segment 104 is therefore disposed between the epidermis and the rectus muscle, and the intraperitoneal tubing segment 102 is disposed within the peritoneum.

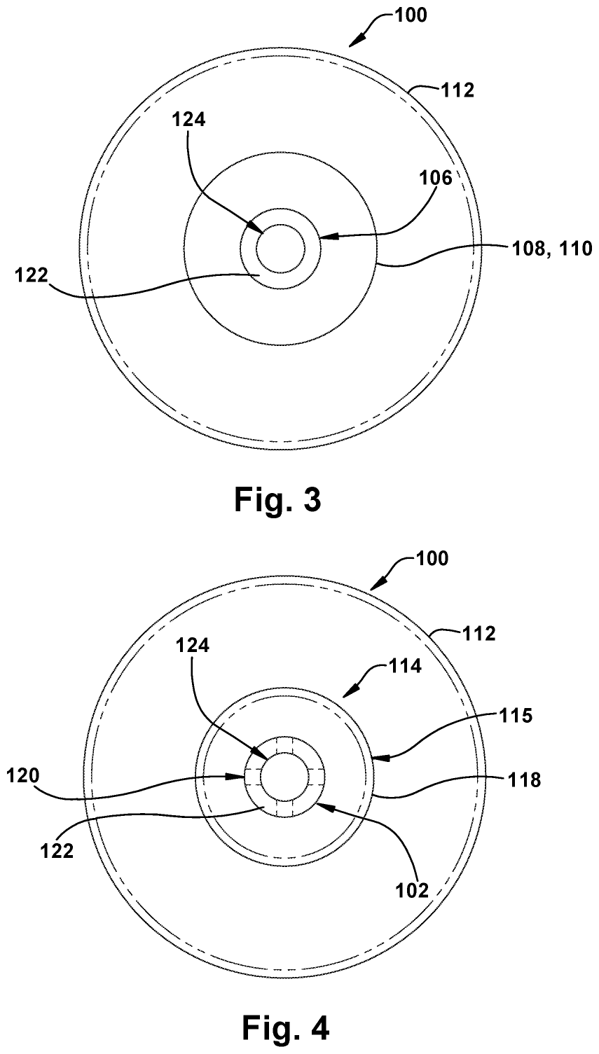

Each of first cuff 108 and second cuff 110, shown in detail in FIG. 3, may be made of a hook and loop (e.g. VELCRO® (registered trademark of Velcro BVBA, Deinze, Belgium)) or woven fiber or material, e.g., a polyester fiber material, which may particularly be DACRON® (registered trademark of Invista North America SARL Corp., Wilmington, Del.) or a similar material. Further, each of first cuff 108 and second cuff 110 may have an outer diameter of about 8 mm and an axial length of about 5 mm to about 15 mm, for example, about 10 mm Additionally, first cuff 108 and second cuff 110 may have a thickness of about 2 mm to about 8 mm, for example, about 5 mm.

Referring back to FIG. 1, a flange 112 is provided adjacent to a distal-facing end of second cuff 110. Flange 112 may be substantially disc-shaped, with a central bore 113 through which catheter 100 passes. Flange 112 may have an outer diameter of about 20 mm to about 30 mm, e g about 25 mm, an axial length of thickness of about 2 mm, and may be made of a hook and loop (e.g. VELCRO® (registered trademark of Velcro BVBA, Deinze, Belgium)) or a woven fiber material, e.g., a polyester fiber material, which may particularly be DACRON® (registered trademark of Invista North America SARL Corp., Wilmington, Del.) or a similar material.

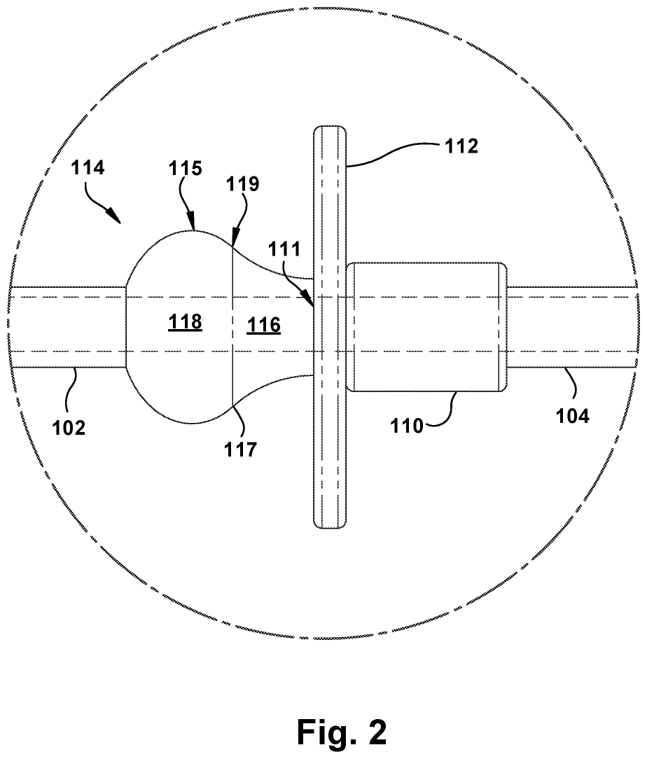

As shown in FIG. 1, and in greater detail in FIG. 2, an anchor 114 is provided adjacent to a distal face of flange 112, for use in surgically affixing a position of the catheter 100 after insertion into a patient. The anchor 114 includes a frustoconical neck member 116 and a bulbous member 118. Bulbous member 118 may have a circular cross section, and may in some embodiments be substantially hemispherical in shape. In other embodiments, bulbous member 118 may be shallower, or less regular in shape than a hemisphere. The bulbous member 118 is coupled at its circular face to the base 117 of the frustum forming the neck member 116. In some embodiments, the frustoconical neck member 116 may more particularly have a concave radially outward-facing surface as shown in FIG. 2. Frustoconical neck member 116 is oriented such that plane 111, which is opposite base 117 of the conical frustum, is disposed adjacent to the distal face of flange 112, and the proximal cross sectional diameter of frustoconical neck member 116 is smaller than the distal cross sectional diameter.

The proximal cross sectional diameter of bulbous member 118 is substantially the same as that of the cross sectional diameter of base 117 of frustoconical neck member 116, such that the profile of the anchor 114 in side view (see FIG. 2) is substantially smooth along the transition 119 from the base 117 of frustoconical neck member 116 to the bulbous member 118.

Distally of transition point 119, the surface of bulbous member 118 curves radially outward, such that the outer cross sectional diameter of bulbous member 118 reaches a widest point 115, at which point the outer cross sectional diameter is about 10 mm to about 15 mm, for example, 12 mm Continuing in a distal direction from the maximum outer diameter at point 115, the outer surface of bulbous member 118 curves radially inward toward intraperitoneal tubing segment 102 of catheter 100. Catheter 100 passes axially through a central bore 113 (FIG. 4) in anchor 114, in similar fashion to flange 112. Anchor 114, which is considered to include frustoconical neck member 116 and bulbous member 118, may have an axial length of about 11-12 mm Collectively, anchor 114, flange 112, and second cuff 110 may have a combined axial length of about 24 mm.

The radially outward facing surface of the frustoconical neck member 116, which as previously noted may be concave in some embodiments, provides a surface for suture or stitch placement during surgical insertion of the device into a patient. The substantially flat or slightly concave shape of the surface promotes sealing between the anchor 114 and the rectus muscle to which it is stitched. This contributes to a shorter healing time required before use in the performance of peritoneal dialysis procedures, and the avoidance of, e.g., occurrences of leaks, infections, and other adverse events.

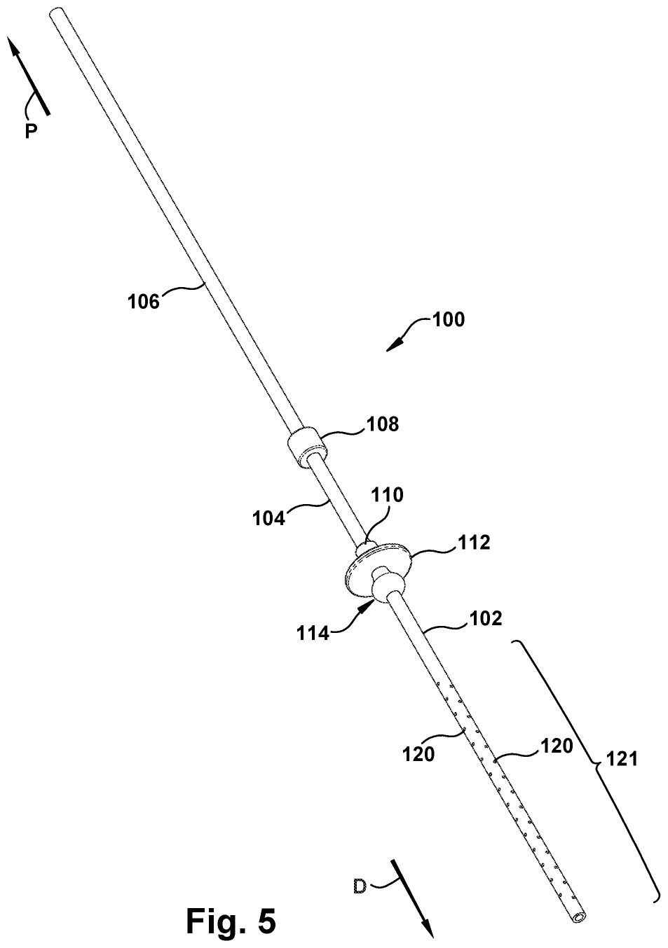

With reference to FIGS. 5-7, in various embodiments catheter 100 includes intraperitoneal tubing segment 102, subcutaneous tubing segment 104, and extraperitoneal tubing segment 106 made of, e.g., 5 mm diameter tubing having a 0.9 mm wall, thereby rendering a 3.2 mm lumen. The tubing may be made of, e.g., platinum silicone, polytetrofluoroethylene, fluorinated ethylene propylene, liquid crystal polymers, polyethylene, polypropylene plastic derivatives, or any other suitable material that includes similar physical/material characteristics. Intraperitoneal tubing segment 102 may further be shaped such that it is substantially straight, with a natural curve rather than e.g., a swan neck.

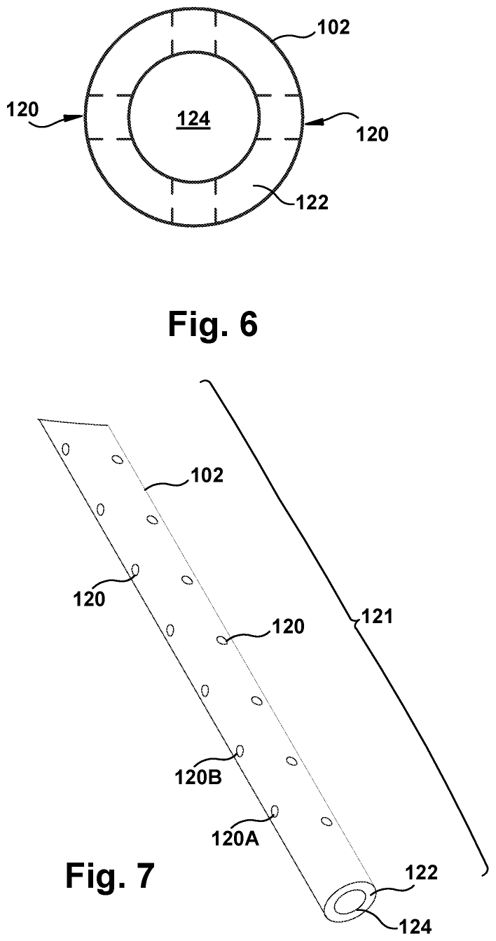

As shown in, e.g., FIGS. 5 and 7, intraperitoneal tubing segment 102 further includes a drain array 121 including a plurality of through holes 120 through the tubing wall 122 (FIG. 7) at a distal end of intraperitoneal tubing segment 102. The through holes 120 may also be referred to as fenestrations or perforations, and extend radially outward through the tubing wall 122 fluidly connecting the central bore 124 (FIG. 6) of the intraperitoneal tubing segment 102 with the peritoneal cavity when inserted into a patient. Each through hole 120 in the drain array 121 may be a nominally 1 mm diameter through-hole in wall 122 of the intraperitoneal tubing segment 102. The drain array 121 in intraperitoneal tubing segment 102 may include approximately sixty such through holes 120.

As shown in FIG. 7, the through holes 120 in the drain array 121 may be arranged in a plurality of lines that are axially and circumferentially spaced around intraperitoneal tubing segment 102. In one embodiment, the through holes 120 may be arranged in four axial lines or rows, with each axial line of through holes 120 being circumferentially spaced by about 90° from each neighboring line of through holes such that a line of through holes 120 may be placed at the 90°, 180°, 270°, and 360° positions. This is illustrated in FIG. 7. Within each axial line, each through hole 120A may be axially spaced from each neighboring through hole 120B within the same line or row by about 7 mm, such that each line or row includes about fifteen through holes evenly spaced over about 105 mm of length of intraperitoneal tubing segment 102. In some embodiments, the four lines or rows of through holes 120 are axially aligned, such that the distal-most through hole 120 in each of the four rows is substantially axially aligned (but circumferentially spaced from) the distal-most through hole 120 in each of the other three rows. Similarly, the proximal-most through hole 120 in each of the four rows is substantially axially aligned (but circumferentially spaced from) the proximal-most through hole 120 in each of the other three rows. In other embodiments, the through holes 120 may be staggered in axial position. For example, a distal-most through hole in a first line or row may be positioned several mm, e.g. 3-4 mm, distally of the distal-most through hole in a second line or row of through holes that is, e.g., 90° around the circumference of intraperitoneal tubing segment 102 from the first line or row. The foregoing arrangements of through holes 120 in drain array 121 are merely exemplary, and variations in the number of lines, axial spacing of holes 120 within lines, circumferential spacing of lines, etc. are all subject to variation, each of which is contemplated as part of the invention.

The advantages associated with the use of an intraperitoneal tubing segment 102 having features as described above is demonstrated in the following example.

### Example 1: Flow Test

A study is conducted to investigate the potential to increase flow-through speed in a peritoneal dialysis catheter, which in turn results in decreased exchange times. An existing benchmark peritoneal dialysis catheter is compared with tubing described above and used in the present device. Characteristics and properties of each are provided in Table 1 below.

TABLE 1
Tubing properties

| | Benchmark | | | |
|---|---|---|---|---|
| | catheter tubing | | Present catheter tubing | |
| Drain array | 44 holes arrayed in four lines, | | 60 holes arrayed in four lines, | |
| | 90° opposed to each other | | 90° opposed to each other | |
| | over a length of 100 mm | | over a length of 105 mm | |
| | (10 | mm spaced) | (7 | mm spaced) |
| Through-hole diameter | 1.0 | mm, nominally | 1.0 | mm, nominally |
| Durometer | 50 | Shore A | 50 | Shore A |
| Force applied by a 12.5 mm | 1.1 | kg | 0.9 | kg |
| diameter probe before tubing | | | | |
| is crushed completely flat | | | | |
| Bend radius without kinking | 10 | mm | 10 | mm |

The use of the same size holes in each specimen preclude hole occlusion by tissue ingrowth from being a factor in this analysis.

The benchmark and subject catheters are each connected to an IV bag filled with precisely 1 liter of water and held straight, suspended over a catch basin. The head height at the start of the test as measured from the distal end of the catheter is 33 inches, and at the end of the test when the IV bag is fully drained is 24 inches.

### Results

The benchmark catheter tubing drained the 1 liter of fluid in a mean time of 2:55 m:ss, measured over three trials. The flow out of the drain holes (44) was consistent with expectations, demonstrating a pressure gradient from the upper to lower holes in that the upper ones drooled while the lower ones supported laminar flow jets. In the same set-up, the subject catheter tubing drained the 1 liter of fluid in a mean time of 2:02 m:ss, measured over three trials. The flow out of the drain holes was consistent with expectations, and consistent with the previous tests. The flow jets were observed to be more forceful than those generated by the benchmark catheter, creating a wider spread in this in vitro test. This is attributed to the slightly shorter flow path through the thinner tubing wall.

### CONCLUSIONS

In vivo, the flow rate through any peritoneal dialysis catheter will be countered by back pressure present within the abdomen. Therefore, the flow rates observed in vitro are merely for comparison, and are not expected to directly reflect in vivo performance. Nonetheless, it is apparent that the increased quantity of through holes (e.g., 60 vs. 44 in the benchmark catheter tubing) results in increased flow rates, and therefore will result in decreased exchange times. Further, neither the use of thinner walled tubing, nor an increase in the number or density of holes perforating it, degrade the performance of the tubing relative to resistance to twisting and kinking. While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A catheter comprising:
an intraperitoneal tubing segment including a drain array, the drain array including a plurality of through holes perforating a wall of a distal end of the intraperitoneal tubing segment;
a subcutaneous tubing segment in fluid connection with a proximal end of the intraperitoneal tubing segment;
an extraperitoneal tubing segment in fluid connection at a distal end thereof with a proximal end of the subcutaneous tubing segment, wherein a proximal end of the extraperitoneal tubing segment is configured for connection to a transfer set;
a first cuff disposed about the catheter between the subcutaneous tubing segment and the extraperitoneal tubing segment;
a second cuff disposed about the catheter between the subcutaneous tubing segment and the intraperitoneal tubing segment;
a flange disposed about the catheter and adjacent to a distal end of the second cuff; and
an anchor including:
a frustoconical neck member having a base, a radially outward facing surface, and a planar surface opposite the base, the frustoconical neck member being positioned such that the planar surface opposite the base is adjacent and substantially parallel to the distal side of the flange, and
a bulbous member coupled to the base of a frustum of the frustoconical neck member.

2. The catheter of claim 1, wherein the frustoconical neck member has a concave frustoconical geometry.

3. The catheter of claim 1, wherein each through hole in the drain array is a nominally 1 mm diameter through-hole in a wall of the intraperitoneal tubing segment.

4. The catheter of claim 1, wherein the plurality of through holes in the drain array comprises about sixty through holes.

5. The catheter of claim 1 wherein the plurality of through holes in the drain array are arranged in four axial lines of through holes, wherein each of the four lines of through holes is circumferentially spaced around the intraperitoneal tubing segment by about 90° from each neighboring line of through holes.

6. The catheter of claim 5, wherein the through holes in each of the four lines of through holes are axially spaced about 7 mm apart.

7. The catheter of claim 6, wherein an axial position of each of the four lines of through holes is staggered with respect to a neighboring line of through holes.

8. The catheter of claim 5, wherein the through holes in each of the four lines are spaced over an axial distance of about 105 mm.

9. The catheter of claim 1, wherein the intraperitoneal tubing segment is substantially straight with a natural curve.

10. The catheter of claim 1, wherein the first cuff and the second cuff each comprise a polyester fiber material.

11. The catheter of claim 1, wherein each of the first cuff and the second cuff has an axial length of about 5 mm to about 15 mm and a thickness of about 2 mm to about 8 mm.

12. The catheter of claim 1, wherein the flange has an outer cross sectional diameter of about 20 mm to about 30 mm.

13. The catheter of claim 1, wherein the anchor has a maximum cross sectional outer diameter of about 10 mm to about 15 mm.

14. The catheter of claim 1, wherein the intraperitoneal tubing segment has a length of about 160 mm.

15. The catheter of claim 1, wherein the subcutaneous tubing segment has a length of about 45 mm.

16. The catheter of claim 1, wherein the anchor has an axial length of about 11-12 mm.

17. The catheter of claim 1, wherein the anchor, the flange, and the second cuff have a combined axial length of about 24 mm.

18. The catheter of claim 1, wherein the intraperitoneal tubing segment comprises a 5 mm diameter tubing with a 0.9 mm wall rendering a 3.2 mm lumen.

19. The catheter of claim 1, wherein the intraperitoneal tubing segment, the subcutaneous tubing segment, and the extraperitoneal tubing segment are a single continuous length of tubing.

20. The catheter of claim 1, wherein the intraperitoneal tubing segment, the subcutaneous tubing segment, and the extraperitoneal tubing segment comprise platinum silicone.

21. An anchor for use in a semi-permanent implantable catheter, comprising:
a frustoconical neck member having a base, a radially outward facing surface, and a planar surface opposite the base, and
a bulbous member coupled to the base of the frustoconical neck member.

22. The anchor of claim 21, wherein the frustoconical neck member has a concave frustoconical geometry, such that the radially outward facing surface is concave.

23. The anchor of claim 21, wherein the bulbous member has a round cross section, and a proximal cross sectional diameter that is substantially the same as the cross sectional diameter of the base of the frustoconical neck member.

24. The anchor of claim 21, wherein a maximum cross sectional diameter of the anchor is reached at a point along the bulbous member between the coupling between the bulbous member and the frustoconical neck member, and the distal end of the bulbous member.

25. The anchor of claim 24, wherein the maximum cross sectional diameter of the anchor is about 12 mm.

26. The anchor of claim 21, further comprising a central axial bore, through which the catheter may pass.

27. The anchor of claim 21, further comprising an axial length of about 11-12 mm.

28. The anchor of claim 21, wherein the radially outward facing surface of the frustoconical neck member is configured to receive sutures during an implantation procedure.

29. A catheter comprising:
an intraperitoneal tubing segment including a drain array, the drain array including about sixty through holes perforating a wall of a distal end of the intraperitoneal tubing segment;
a subcutaneous tubing segment in fluid connection with a proximal end of the intraperitoneal tubing segment;
an extraperitoneal tubing segment in fluid connection at a distal end thereof with a proximal end of the subcutaneous tubing segment, wherein a proximal end of the extraperitoneal tubing segment is configured for connection to a transfer set;
a first cuff disposed about the catheter between the subcutaneous tubing segment and the extraperitoneal tubing segment;
a second cuff disposed about the catheter between the subcutaneous tubing segment and the intraperitoneal tubing segment;
a flange disposed about the catheter and adjacent to a distal end of the second cuff; and
an anchor adjacent to the distal side of the flange.

30. The catheter of claim 29, wherein each through hole in the drain array is a nominally 1 mm diameter through-hole in a wall of the intraperitoneal tubing segment.

31. The catheter of claim 29, wherein the drain array includes a plurality of through holes arranged in four axial lines of through holes, wherein each of the four lines of through holes is circumferentially spaced around the intraperitoneal tubing segment by about 90° from each neighboring line of through holes.

32. The catheter of claim 31, wherein the through holes in each of the four lines of through holes are axially spaced about 7 mm apart.

33. The catheter of claim 32, wherein an axial position of each of the four lines of through holes is staggered with respect to a neighboring line of through holes.

34. The catheter of claim 31, wherein the through holes in each of the four lines are spaced over an axial distance of about 105 mm.

35. The catheter of claim 29, wherein the intraperitoneal tubing segment is substantially straight with a natural curve.

36. The catheter of claim 29, wherein the first cuff and the second cuff each comprise a polyester fiber material.

37. The catheter of claim 29, wherein each of the first cuff and the second cuff has an axial length of about 10 mm and a thickness of about 5 mm.

38. The catheter of claim 29, wherein the flange has an outer cross sectional diameter of about 20 mm to about 30 mm.

39. The catheter of claim 29, wherein the anchor has a maximum cross sectional outer diameter of about 12 mm.

40. The catheter of claim 29, wherein the intraperitoneal tubing segment has a length of about 160 mm.

41. The catheter of claim 29, wherein the subcutaneous tubing segment has a length of about 45 mm.

42. The catheter of claim 29, wherein the intraperitoneal tubing segment comprises a 5 mm diameter tubing with a 0.9 mm wall rendering a 3.2 mm lumen.

43. The catheter of claim 29, wherein the intraperitoneal tubing segment, the subcutaneous tubing segment, and the extraperitoneal tubing segment are a single continuous length of tubing.

44. The catheter of claim 29, wherein the intraperitoneal tubing segment, the subcutaneous tubing segment, and the extraperitoneal tubing segment comprise platinum silicone.
